(19) United States
(12) Patent Application Publication
(10) Pub. No.: US 2004/0267315 A1
(43) Pub. Date: Dec. 30, 2004

(54) Braided gold suture and method of use

(76) Inventors: Bradley R. Wolf, Cincinnati, OH (US); Igor A. Bogin, Alma-Aty (KZ); Ruben A. Bogin, Westerville, OH (US)

Correspondence Address: MARK P. LEVY, ESQ., THOMPSON HINE, LLP, 2000 COURTHOUSE PLAZA NE, 10 W. SECOND STREET, DAYTON, OH, 45402-1758, US

(21) Appl. No.: 10/606,192

(22) Filed: Jun. 25, 2003

(51) Int. Cl. A61L 17/00; A61F 2/10

(52) U.S. Cl. 606/230; 623/15.12

(57) ABSTRACT

A braided suture for skin lifting comprising gold threads and bioabsorbable threads, wherein the gold threads and bioabsorbable threads are braided to form a braided composite suture. A method of skin lifting providing a braided composite suture comprising gold threads and bioabsorbable threads and implanting the braided composite suture intradermally at a plurality of skin regions.

### BACKGROUND

This invention relates to cosmetic and plastic surgery and can be used for lifting skin and underlining soft tissues for eliminating age changes, improving circulation and increasing the amount of the collagen component of the skin.

One method of surgical removal of wrinkles on the face and neck is by facelift, or rhytidectomy, designed to excise excess skin and reposition the sagging soft tissue of the face. The standard incision extends from a point in the temporal scalp approximately 6 cm. superior to the root of the helix along the anterior margin of the ear around the lobe and along the posterior sulcus of the ear, terminating in the hear-bearing scalp. The skin of the face and neck is then dissected from the underlying muscles of facial expression. The SMAS (superficial muscular aponeurotic system) is tightened with the skin flap or as a separate layer by dissecting or by plicating it to itself. (S. Schwartz, R. Wood, M. Jurkewicz “Principles of Surgery” Chpt. Plastic and Reconstructive Surgery, Seventh Edition, McGRAW-HILL, N.Y., et al. pp.2137-2138). The disadvantages of this procedure are trauma, and the risk of complications: hematoma formation in approximately 4 percent, facial nerve injury in less than 1 percent and delayed wound healing may be seen in the posterior auricular incisions, especially in patients who smoke.

Another method for smoothing facial folds and wrinkles uses the subcutaneous introduction of dermal autotransplants into a specifically formed pocket in the skin (see U.S. Pat. No. 5,397,352 C1-A 61 F 2/02, published 1995). The drawbacks to this method are trauma and low efficiency. Shortly after the procedure, the implanted areas decrease and the skin folds appear again.

Yet another method for skin lifting utilizes the subcutaneous implantation into the desired area of an inert material consisting of liquid silicone. Rees T. D., Plastic Reconstruction Surgery of the Face and Neck, 1977, volume 1, “Silicone Injection therapy in Atrophy of the Face”, pgs. 247-250. There are several disadvantages to this particular procedure which include: low efficiency due to a rapid migration into adjacent tissue, reduction in volume, slacking, post-operational after-effects including non-specific inflammation and allergic reactions. Also silicone fails to provide a prolonged and stable cosmetic effect.

Sutures can also be used in cosmetic and plastic surgery for skin lifting. Sutures have been used to close wounds, secure damage tendons and hold tissue together to support healing and re-growth. Many types of sutures have been used in various surgical operations; sutures can be monofilament or braided, or be of other geometrical properties. Sutures can also be non- absorbable or bioabsorbable. Non-absorbable sutures are used in surgical operations which require the sutures to be in place to hold the tissue or wound together for a longer period of time, whereas bioabsorbable sutures are absorbed into the body after a limited period of time and are used in operations that do not require the suture to be in place for a long period. Nevertheless, the problem with non-absorbable sutures is that they can still have some slow degradation which causes a decrease in tensile strength. Additionally, they frequently become the source of tissue reaction and infection/inflammation. This could result in excessive scar formation or wound or tissue dehiscence. The use of bioabsorbable sutures are also limited and not an option in wounds requiring high tension because after degradation they leave underlining tissue without support which may cause the skin scar to increase in size.

Sutures with braids have been described and used successfully in skin lifting, but the short span of studies and significant failure rate—10 percent, leave room for improvement and long term studies indicate potential side effects. Leaving in polymer, non-absorbable sutures, especially in cosmetically significant areas, causes significant long lasting tissue reaction with a high incidence of infection. Also, the continuous degradation of the suture reduces the lifting effects. Accordingly, it is desirable to have a suture that can be used in skin lifting and can be left in place without side effects. The use of gold sutures is particularly useful because gold is an inert material compatible with the body and almost never cause infection or inflammation when implanted in body tissue and left in place.

U.S. Pat. No. 6,086,578 discloses a type of suture for skin lifting consisting of golden filament. The method disclosed in this skin rejuvenation process is comprised of the subcutaneous implantation of golden threads in the rejuvenation area. The golden threads are implanted along and/or across wrinkles or skin folds found in the specific rejuvenation area in a way so they catch skin areas located behind a specific rejuvenation area. The input and output sites of the golden threads are the intersection points of vertical and horizontal lines (forming a grid or net) drawn across the rejuvenation area. The length of these lines is longer than the wrinkles found in the rejuvenation area. Nevertheless, the suture is unbraided and fragile. Another disadvantage from using the unbraided threads in this procedure is that the threads are located behind a skin fold area, along them or in the grid pattern, which does not producing any particular direction of skin traction and therefore not a skin lifting procedure. Partial wrinkle correction is only achieved by the effect of fibroblast proliferation that promotes collagen and elastin production, which provides a tightening effect. In addition, the high fragility of the golden thread produces a high failure rate of the procedure and prolongs the learning curve for surgeons. Even reinforcing them with absorbable non braided filaments usually does not improve strength significantly, but actually leaves wider passage tunnels resulting in more trauma and thereafter increasing possible complications as infections, hematoma and inflammation.

### SUMMARY

The invention is directed to a braided suture and improved method of use which overcomes the disadvantages in the prior procedure. The invention is a suture for skin lifting comprising gold threads and bioabsorbable threads, wherein the gold threads and bioabsorbable threads are braided to form a braided composite suture. The diameter (thickness) of the suture is determined by the number of threads that are braided. The degree of thickness of the suture will be used with respect to the thickness of the patient's skin. The bioabsorbable threads add tensile strength and become absorbed in the body while the gold threads retain the zigzag braided configuration thereby enhancing the amount of collagen production around the threads. The collagen production lifts the skin and helps remove wrinkles. The braid structure of the suture also creates grooves which provide traction when implanted and allows the surgeon to lift the skin thereby aiding in the wrinkle correction.

In an embodiment of the invention, a method of use for the braided suture is the intradermal implantation of the suture for lifting of the skin. By positioning the braided sutures intradermally where more cells are available for collagen synthesis, the production of collagen is increased resulting in a more efficient skin lifting process. The method can be used to lift the skin in areas on the face, neck, arms, legs and body.

### BRIEF DESCRIPTION OF THE DRAWINGS

FIG. 1A is a view of the braided composite suture comprising a gold thread and a bioabsorbable attached to a needle.

FIG. 1B is a graph showing the geometrical measurements of the braided suture.

FIG. 2 is a view of the braided suture being implanted intradermally.

FIG. 3 is an embodiment of the method of the invention wherein the lines are drawn for applying the braided sutures intradermally to the various areas of the facial and neck region.

### DETAILED DESCRIPTION OF THE INVENTION

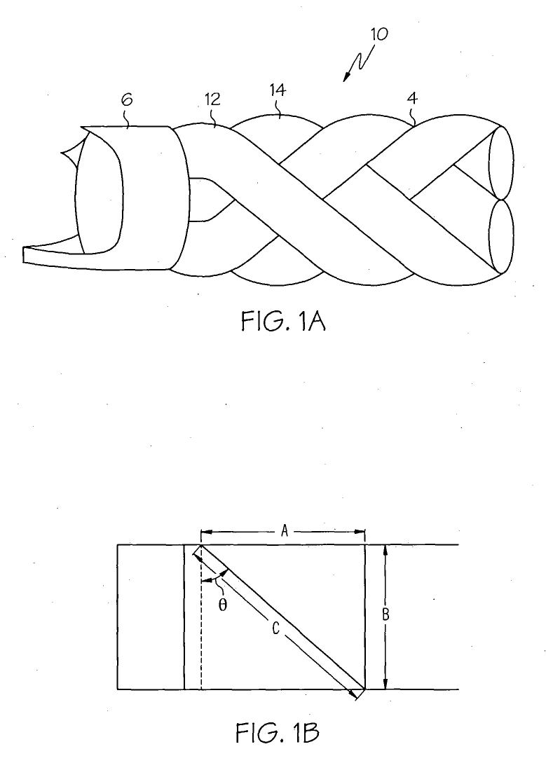

The invention is directed to a braided suture and its method of use in plastic and cosmetic surgery. Referring to FIG. 1A, the suture is a composite suture comprising braided gold and bioabsorbable threads for the intracutaneous implantation in the skin. The use of this braided suture is particularly useful for skin lifting.

In a preferred embodiment, the bioabsorbable threads are polymeric threads which become absorbed in the body in approximately 45-60 days. In one manifestation of the invention, the polymeric threads are formed of polyglactin. Other examples of bioabsorbable sutures include monocryl, chromic gut, surgigut, and plain gut sutures. The bioabsorbable threads may either be coated or non-coated.

Gold threads are useful as sutures because gold is an inert material compatible with the body and almost never cause infection or inflammation when implanted in body tissue and left in place. The advantage of gold threads is that they can remain in the body and do not need to be removed. This is especially useful in skin lifting operations where the gold threads are implanted under the skin wrinkles for the purpose of producing collagen around the threads, thereby improving tissue rigidity and lifting the skin. Typically, the gold alloy is above 22 carats.

The suture may be braided very tightly or less tightly as desired; however, the suture is only required to be braided tight enough to maintain the braid. A tighter braid creates more zigzag pattern for the gold thread, but the depth of the grooves decrease once the bioabsorbable thread is absorbed, which enhances the production of collagen by creating more surface area for the gold thread as previously mentioned. FIG. 1A shows the braided composite suture 10 comprising a gold thread 12 and a bioabsorbable thread 14 attached to a needle 6. Additionally, grooves 4 are created by the braided structure of the suture 10. FIG. 1B shows the geometrical measurements of the suture. The pace of the braided structure is represented by A, the diameter of the suture is represented by B, the length of the thread in a pace A is represented by C and the braid angle is represented by θ. Typically the suture is braided such that the braid angle θ is 45°. A more tightly braided suture will have a braid angle less than 45° while a looser braided suture will have a braid angle greater than 45°. Additionally, the tighter the braid of the suture, the less the length of the thread C in a pace A.

Because skin thickness will differ with each patient as well as in different locations of each patient, the thickness of the suture will be used with respect to the thickness of the patient's skin. In an embodiment of the invention, the suture can range from 2 to 10 gauge in thickness. Generally, a suture with 10 gauge thickness would comprise about 3 threads, a suture between 4 to 6 gauge would comprise 5 to 6 threads and a suture between 2 to 4 gauge would comprise about 9 threads.

Braiding the bioabsorbable threads and the gold threads into a composite suture results in many advantages over the use of the gold threads alone in skin lifting. After implanting braided suture, the bioabsorbable thread becomes absorbed in the body, while the gold thread stays in place. Physiologically, this is advantageous since the braided gold threads remain in place in a zigzag pattern once the bioabsorbable thread is absorbed because of the initial braid structure of the gold thread that was implanted. The zigzag pattern increases the total length of the gold thread by up to 40% over a straight non-braided gold suture. As a result, this increases the amount of fine collagen slivers produced in the scar tissue which enhances the lifting of the wrinkles.

Additionally, the braided structure of the suture has several advantages over a regular suture in this procedure. The braided structure provides the suture with more flexibility to maneuver in surgical applications and furthermore, adds tensile strength to the suture which allows use in higher tension procedures. The braids also allows the sutures to be knotted and tied. Furthermore, the grooves from the braided configuration creates frictional communication as the suture is passed through the intraderma layer providing adequate traction, thereby allowing the surgeon to physically “lift” the wrinkle by applying the desired tension while passing the suture intradermally. Accordingly, the braided suture helps wrinkle correction by the dual means of enabling the surgeon to lift the wrinkle and enhancing production of collagen around the zigzagged gold thread after implantation. Thus, the braided structure is beneficial over a straight suture both from a physical and physiological standpoint.

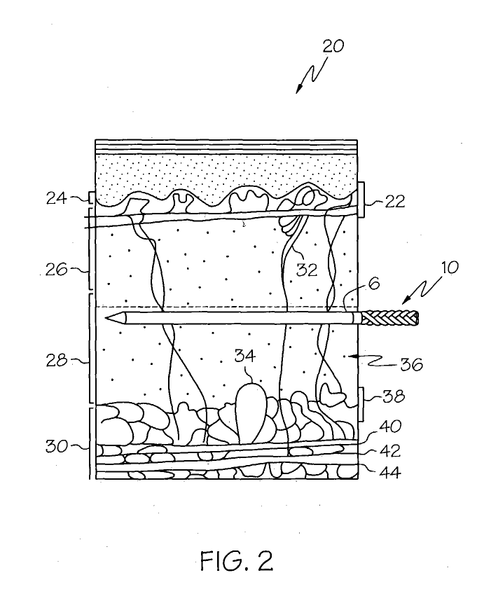

This invention is also directed to a method of implanting the braided suture intradermally for skin lifting procedures. By positioning the sutures intradermally, the efficiency of skin lifting is increased. Because the suture is positioned intradermally where the most fibroblasts are located, there are more cells available for regulating collagen synthesis and degradation, as well as myofibroblasts which contract to form scar tissue around the suture. This results in a more efficient skin lifting process than the subdermal positioning of the sutures described in the prior art. FIG. 2 shows the bioabsorbable/gold composite suture being implanted intradermally. The layers and formations within the skin 20 include the papillary plexus 22, the stratum germativum 24, the papillary derma 26, the reticular derma 28, the dermal-subcutaneous plexus 38 and the subcutaneous layer 30 wherein the subcutaneous layer 30 includes a subcutaneous artery 40, vein 42 and nerve 44. The skin 20 also includes nerve endings 32, 34 and fibroblasts 36. The braided suture 10 is implanted by inserting the needle 6 intradermally.

In a preferred embodiment of the method, lines are first drawn with a sterile surgical pencil along the skin wrinkle and then gradually turned toward the direction of desired traction of the skin. A bendable straight needle with a triple-edged sharpened point between 5 to 12 cm in length is introduced into the skin and advanced along the drawn line intradermally. The depth and direction of the needle are controlled with the fingers. At the end of the drawn line, the needle tip is taken through the skin, and the suture is pulled through the formed passage. The end of the suture is cut off at the level of the skin. Next, the needle is held such that the desired tension is produced straightening any small plications that are formed and the needle end of the suture is cut. Because of the grooves on the braided sutures and its communication with the surrounding intradermal tissues, the suture is held in position. In a further embodiment of the method, the length of the needle is greater than the distance between the thread input site and the thread output site.

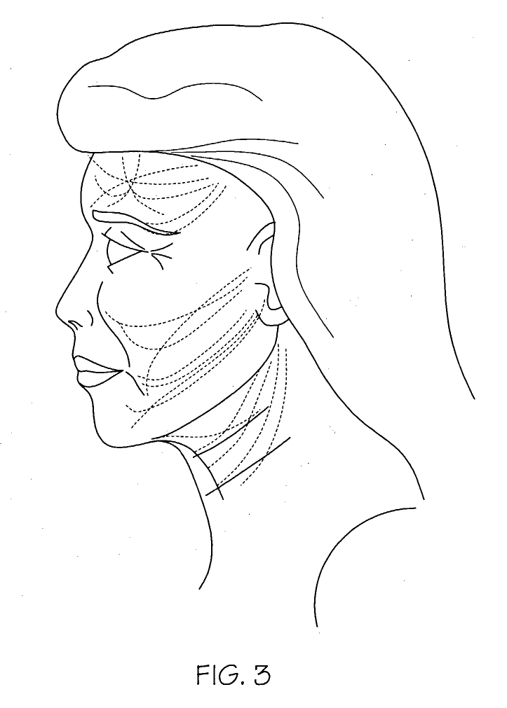

The lines that are drawn for implanting the sutures include areas of skin located on the face and body where wrinkles and soft tissue ptosis are present. The areas on the face include the nasolabial folds, the forehead folds, the lateral angle of the eyes and the chin. The areas on the body include the front of the neck, the inner and outer areas of the arms, the thighs, the breasts and the stomach. The lines can be drawn along the wrinkles and end on areas where exit sites are most unnoticeable. The lines can also be drawn so that they cross the wrinkles. The number of lines drawn depend on several factors including the amount of ptosis, the thickness of the skin and the area of lifting. Furthermore, the direction and curvature of the lines depend on the depth of the wrinkles, direction of the folds, skin elasticity and location of the region. FIG. 3 shows an embodiment of the method of the invention wherein the lines are drawn for applying sutures intradermally to the various areas of the facial and neck region.

What is claimed:

1. A braided suture for skin lifting comprising:
gold threads and bioabsorbable threads, wherein the gold threads and bioabsorbable threads are braided to form a braided composite suture.

2. The suture of claim 1 wherein the suture has a thickness in the range of 2 to 10 gauge.

3. The suture of claim 2 wherein the suture is braided with 3 to 9 threads.

4. The suture of claim 1 wherein the suture comprises 1 to 5 gold threads.

5. The suture of claim 1 wherein the suture comprises 1 to 5 bioabsorbable threads.

6. The suture of claim 1 wherein the suture comprises 50% gold threads and 50% bioabsorbable threads.

7. The suture of claim 1 wherein the bioabsorbable threads can be selected from polymeric threads and non-polymeric threads.

8. The suture of claim 1 wherein the gold threads are from different gold alloys above 22 carats.

9. The suture of claim 1 wherein the suture is attached to different types of needles.

10. The suture of claim 1 wherein the suture has a braid angle of 45 degrees.

11. A method of skin lifting comprising:
providing a braided composite suture comprising gold threads and bioabsorbable threads; and
implanting the braided composite suture intradermally at a plurality of skin regions.

12. The method of claim 11 wherein the plurality of skin regions are located in specific skin fold areas.

13. The method of claim 11 wherein the plurality of skin regions are areas of desired traction directions outside of specific skin fold areas and wherein the sutures are implanted in continuity with the specific skin fold areas.

14. The method of claim 13 wherein a desired tension is applied lift the skin fold areas to help wrinkle correction.

15. The method of claim 11 wherein the braided composite suture is ultra thin.

16. The method of claim 11 wherein a length of a needle is greater than the distance between a thread input site and a thread output site.
